(19) United States
(12) Patent Application Publication
(10) Pub. No.: US 2023/0190558 A1
(43) Pub. Date: Jun. 22, 2023

(54) MOBILE ISOLATION WARD

(71) Applicants: Jin Yeong KIM, Gimhae-si (KR); Youn Suk LEE, Gimhae-si (KR)

(72) Inventors: Jin Yeong KIM, Gimhae-si (KR); Youn Suk LEE, Gimhae-si (KR)

(21) Appl. No.: 17/555,079

(22) Filed: Dec. 17, 2021

(51) Int. Cl. A61G 10/00; A61G 7/05; A61L 9/20; A61L 2/10; A61L 2/24; E04H 1/12

(52) U.S. Cl. CPC A61G 10/005; A61G 7/05; A61L 9/20; A61L 2/10; A61L 2/24; E04H 1/125; A61G 2203/30; A61G 2203/46; A61L 2202/25; A61L 2202/11; A61L 2202/14; A61L 2202/16; A61L 2209/14; A61L 2209/111

(57) ABSTRACT

Disclosed herein is a mobile isolation ward including: a main body configured such that a door is installed in a front thereof, a fixed lighting window is installed in a right side thereof, and a light is installed at the center of a ceiling thereof; a ceiling air conditioner installed in the right side of the ceiling of the main body, and configured to control the temperature and the humidity inside the main body; an air purifier provided on one side of the inside of the main body, and configured to purify contaminated air inside the main body via a filter; and a blower configured to selectively introduce and discharge air. The junctions of the lighting window and the main body are finished with silicone. The main body is made of a glass fiber reinforced plastic material. The door and the lighting window are made of a transparent acrylic material.

### BACKGROUND

### 1. Technical Field

The present invention relates generally to a mobile isolation ward, and more particularly to a mobile isolation ward in which a main body made of a glass fiber reinforced plastic material in a compact size is provided, the inner surface of the main body is surface-treated with an eco-friendly polymer paint, a fixed lighting window finished with silicone is provided in the right side of the main body, and an air purifier equipped with a 13-level or higher high-efficiency particulate air (HEPA) filter is installed on one side of the inside of the main body.

### 2. Description of the Related Art

Recently, the rate of increase in the number of patients infected with COVID-19 has risen sharply, and the locally prevalent community infection is spreading across the country. Due to this, intensive care beds and general beds used to treat COVID-19 patients are substantially filled up, resulting in a large-scale shortage of beds.

Due to this situation, COVID-19 patients cannot be isolated and treated and the number of patients waiting at home increases, so that there occurs a crisis situation in which the problem of treating these patients arises and the possibility of family-to-family transmission increases. As a result, the tragedy in which many severe COVID-19 patients die alone at home because they do not receive timely treatment is becoming a reality.

Accordingly, in order to isolate and treat a large number of patients in preparation for an infectious disease such as Corona 19, it is necessary to secure sufficient accommodation spaces such as auditoriums and gymnasiums in the community and to provide a number of mobile isolation wards in the accommodation spaces.

However, since the existing mobile isolation wards have a large size and a number of pieces of unnecessary equipment are installed therein, there is a problem in that it takes a lot of time and money to manufacture such mobile isolation wards.

Furthermore, the existing mobile isolation wards have a problem in that contaminated air inside the mobile isolation wards may leak to the outside through a gap formed in connection with a door or a window. In addition, there is a problem in that the existing mobile accounting wards cannot completely filter out droplets contained in contaminated air therein.

Moreover, the existing mobile isolation wards do not have a system for controlling an environment inside the ward according to a patient's condition, so that there is a problem in that it is not possible to provide a customized medical environment optimized for each patient.

### Related Art Document

Patent document: Korean Patent No. 10-1646524

### SUMMARY

The present invention has been conceived to overcome the above-described problems, and an object of the present invention is to provide a mobile isolation ward which is provided with a main body made of a glass fiber reinforced plastic material in a compact size.

Another object of the present invention is to provide a mobile isolation ward in which the inner surface of a main body is surface-treated with an eco-friendly polymer paint.

Another object of the present invention is to provide a mobile isolation ward which is provided with a fixed lighting window finished with silicone in the right side of the main body.

Another object of the present invention is to provide a mobile isolation ward in which an air purifier equipped with a 13-level or higher HEPA filter that filters out 99.95% or more of 0.3 μm-sized particles is installed on one side of the inside of a main body.

Another object of the present invention is to provide a mobile isolation ward which is provided with a system for controlling an environment inside a main body according to the personal information, type, external temperature and humidity of a patient, so that a customized medical environment optimized for each patient may be provided.

According to an aspect of the present invention, there is provided a mobile isolation ward including: a main body configured such that a door through which a patient can enter and exit is installed in the front thereof, a fixed lighting window is installed in the right side thereof, and a light is installed at the center of the ceiling thereof; a ceiling air conditioner installed in the right side of the ceiling of the main body, and configured to control the temperature and the humidity inside the main body; an air purifier provided on one side of the inside of the main body, and configured to purify contaminated air inside the main body via a filter installed therein; and a blower communicating with both opposite sides of the main body, and configured to selectively introduce external air and discharge air purified by the air purifier; wherein the junctions of the lighting window and the main body are finished with silicone; wherein the main body is made of a glass fiber reinforced plastic material; and wherein the door and the lighting window are made of a transparent acrylic material.

A hospital bed configured to allow a patient to lay thereon may be provided in the lower side of the inside of the main body, a storage cabinet may be disposed on the left side of a portion over the hospital bed, a mattress may be disposed on the right side of the cabinet, and the filter may be a 13-level or higher high-efficiency particulate air (HEPA) filter.

The inner surface of the main body may be surface-treated with an eco-friendly polymer paint, and a plurality of ultraviolet sterilization lamps configured to sterilize the inside of the main body may be provided on one side of the inside of the main body.

The mobile isolation ward may further include: patient monitoring equipment configured to check the patient's condition in real time by continuously monitoring biosignals such as the patient's electrocardiogram, oxygen saturation, body temperature, blood pressure, and respiration rate; a sensor unit configured to measure environment-related data inside or outside the main body; and a controller configured to control an internal environment of the main body to provide a customized medical environment optimized for each patient based on data measured by the sensor unit. The sensor unit may include: a first temperature sensor configured to measure an actual temperature inside the main body; a first humidity sensor configured to measure an actual humidity inside the main body; a second temperature sensor configured to measure an actual temperature outside the main body; a second humidity sensor configured to measuring an actual humidity outside the main body; an illuminance sensor configured to measure an actual illuminance inside the main body; an opening/closing sensor configured to detect whether the door is opened or closed, and to generate an opening signal or a closing signal; an odor sensor configured to measure an actual concentration of an odor-causing material floating in an inner space of the main body; a bacteria detection sensor configured to detect an actual concentration of bacteria inside the main body; a gas concentration sensor configured to measure an actual concentration of carbon dioxide in the main body; a fine dust sensor configured to measure an actual concentration of fine dust inside the main body; and a noise sensor configured to measure an actual noise inside the main body.

The controller may include: an input unit configured to receive basic information of the patient, a reference temperature section and reference humidity section for each type of patient, a set temperature section based on an actual temperature outside the main body, and a set humidity section based on an actual humidity outside the main body; a selection mode configured such that the type of patient suitable for a disease of the patient accommodated in the main body is selected; an alarm unit configured to generate an alarm sound when an emergency situation occurs; and an air condition control unit configured to calculate a standard temperature and standard humidity suitable for the patient based on the basic information of the patient, the type of patient, and the actual temperature and actual humidity outside the main body, and to control the ceiling air conditioner according to the standard temperature and the standard humidity. The basic information may be the height, weight, and age of the patient. The air condition control unit may be further configured to: calculate obesity of the patient based on the height and weight of the patient, and calculate a preliminary standard temperature inside the main body suitable for the patient by taking into consideration the age, obesity, and body temperature of the patient in an integrated manner; calculate the preliminary standard temperature inside the main body as a lower value as the age of the patient is younger or the obesity of the patient is higher, and calculate the preliminary standard temperature inside the main body as a lower value as the body temperature of the patient is higher; calculate an overlap temperature section in which temperature values overlap each other by comparing the reference temperature section based on the type of patient and the set temperature section based on the actual temperature outside the main body; set the preliminary standard temperature as the standard temperature suitable for the patient when the preliminary standard temperature calculated according to the basic information of the patient falls within the overlap temperature section, set an upper limit of the overlap temperature section as the standard temperature when the preliminary standard temperature calculated according to the basic information of the patient exceeds the overlap temperature section, and set a lower limit of the overlap temperature section as the standard temperature when the preliminary standard temperature calculated according to the basic information of the patient is lower than the overlap temperature section; and perform control so that the ceiling air conditioner operates until the actual temperature inside the main body becomes the standard temperature.

The air condition control unit may be further configured to: calculate an overlap humidity section in which humidity values overlap each other by comparing the reference humidity section based on the type of patient and the set humidity section based on the actual humidity outside the main body; and calculate an average value of upper and lower limits of the overlap humidity section, set the average value as the standard humidity, and perform control so that the ceiling air conditioner operates until the actual humidity inside the main body reaches the standard humidity.

The controller may include: an illuminance control unit configured to, when the actual illuminance inside the main body measured by the illuminance sensor is lower than a preset reference illuminance, perform control so that an illuminance of the light increases until the actual illuminance inside the main body reaches the reference illuminance; a wind speed control unit configured to, when the actual wind speed inside the main body measured by the wind speed sensor is lower than a preset reference wind speed, perform control so that wind speeds of the air purifier and the blower increase until the actual wind speed inside the main body reaches the reference wind speed; an atmospheric control unit configured to, when the actual concentration of odor-causing material measured by the odor sensor exceeds a preset reference concentration, perform control so that the blower operates for a preset reference time and also perform control so that the air purifier operates until the actual concentration of the odor-causing material inside the main body decreases to a value equal to or lower than the reference concentration; and a noise control unit configured to, when the actual noise inside the main body measured by the noise sensor exceeds a preset reference level and the opening signal indicative of opening of the door is generated by the opening/closing sensor, perform control so that the door is closed and the alarm unit generates an alarm sound. The reference illuminance may be the illuminance inside the main body that is set to vary according to time.

The illuminance control unit may be further configured to perform control so that the illuminance of the light increases until the actual illuminance inside the main body becomes 1.2 times the reference illuminance in response to the opening signal indicative of the opening of the door generated by the opening/closing sensor. The atmospheric control unit may be further configured to: when the actual concentration of bacteria inside the main body detected by the bacteria detection sensor exceeds a preset concentration, perform control so that the air purifier operates until the actual concentration of bacteria in the main body decreases to a value equal to or lower than the set concentration; when the actual concentration of carbon dioxide inside the main body measured by the gas concentration sensor exceeds a preset upper limit concentration, perform control so that the blower operates until the actual concentration of carbon dioxide inside the main body decreases to a value equal to or lower than the preset upper limit concentration; and, when the actual concentration of fine dust inside the main body measured by the fine dust sensor exceeds a preset limit concentration, perform control so that the air purifier operates until the actual concentration of fine dust inside the main body decreases to a value equal to or lower than the limit concentration.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a perspective view of a mobile isolation ward according to the present invention;

FIG. 2 is a front view of the mobile isolation ward shown in FIG. 1;

FIG. 3 is a right side view of the mobile isolation ward shown in FIG. 1;

FIG. 4 is a block diagram of a sensor unit;

FIG. 5 is a block diagram of a controller;

FIG. 6 is a block diagram illustrating a process in which an air condition control unit performs control so that the temperature inside a main body reaches a standard temperature;

FIG. 7 is a block diagram illustrating a process in which the air condition control unit performs control so that the actual humidity inside the main body reaches a standard humidity;

FIG. 8 is a block diagram illustrating a process in which an illuminance control unit performs control so that the actual illuminance inside the main body reaches a reference illuminance;

FIG. 9 is a block diagram illustrating a process in which a wind speed control unit performs control so that the actual wind speed inside the main body reaches a reference wind speed;

FIG. 10 is a block diagram illustrating a process in which an atmospheric control unit performs control so that the actual concentration of odor-causing material inside the main body becomes equal to or lower than a reference concentration; and

FIG. 11 is a block diagram illustrating a process in which a noise control unit reduces the noise inside the main body.

### DETAILED DESCRIPTION

Embodiments of the present invention will be described below with reference to the accompanying drawings in order to describe the present invention in detail so that those of ordinary skill in the art to which the present invention pertains can easily implement the technical spirit of the present invention.

However, the following examples are merely examples intended to help the understanding of the present invention, but are not intended to reduce or limit the scope of the present invention. In addition, the present invention may be embodied in various different forms and is not limited to the embodiments described herein.

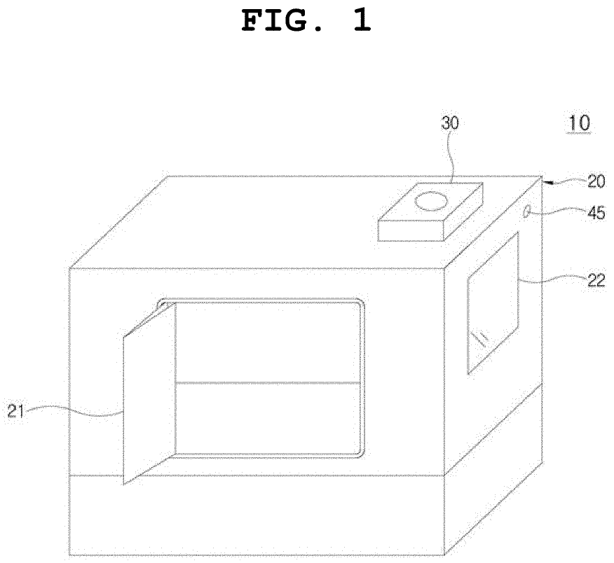

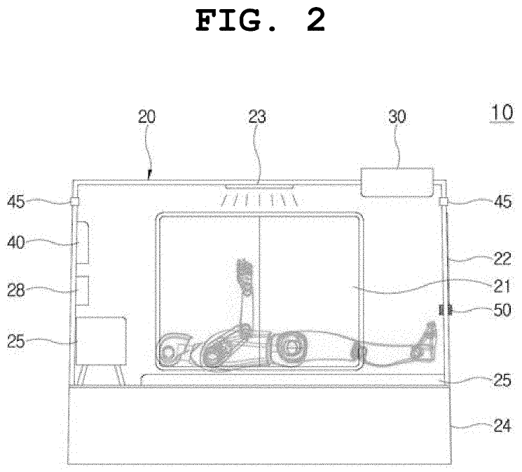

FIG. 1 is a perspective view of a mobile isolation ward 10 according to the present invention, and FIG. 2 is a front view of the mobile isolation ward 10 shown in FIG. 1.

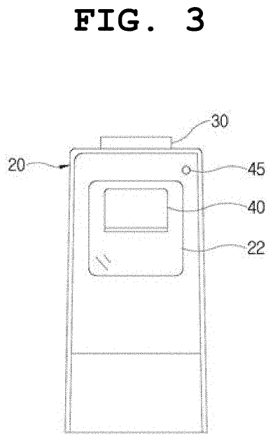

FIG. 3 is a right side view of the mobile isolation ward 10 shown in FIG. 1.

Referring to FIGS. 1 to 3, the mobile isolation ward 10 according to the present invention is configured to include a main body 20, a ceiling air conditioner 30, an air purifier 40, and a blower 45.

First, a door 21 configured to allow a patient to enter and exit and to be automatically opened and closed is installed in the front side of the main body 20, and a fixed lighting window 22 is installed in the right side of the main body 20. In addition, a light 23 is installed at the center of the ceiling of the main body 20.

The main body 20 is made of a fiberglass reinforced plastic (FRP) material that has desirable corrosion resistance, heat resistance, and durability and is easy to handle. The inner surface of the main body 20 is surface-treated with an eco-friendly polymer paint to prevent diseases such as dermatitis, respiratory disease, and carcinogenesis in advance.

In addition, the door 21 is formed such that the inside of the main body 20 is completely blocked to prevent the inflow of external air when it is closed.

In addition, the junctions of the lighting window 22 and the main body 20 are finished with silicone. In addition, the door 21 and the lighting window 22 are made of a transparent acrylic material so that a patient's health condition can be checked from time to time.

In addition, a hospital bed 24 configured to allow a patient to lay thereon is provided in the lower side of the inside of the main body 20, a storage cabinet 25 is disposed on the left side of a portion over the hospital bed 24, and a mattress 26 is disposed on the right side of the cabinet 25.

In addition, a plurality of ultraviolet sterilization lamps (not shown) configured to sterilize the inside of the main body 20 is provided on one side of the inside of the main body 20.

Meanwhile, patient monitoring equipment 28 is separately provided inside the main body 20. In this case, the patient monitoring equipment 28 is a medical device that can check a patient's conditions in real time by continuously monitoring biosignals such as the patient's electrocardiogram, oxygen saturation, body temperature, blood pressure, respiration rate, etc. using sensors.

In addition, the ceiling air conditioner 30 is installed on the right side of the ceiling of the main body 20 and serves to control the temperature and humidity of the inside of the main body 20.

In addition, the air purifier 40 is provided on one side of the inside of the main body 20 and serves to purify contaminated air inside the main body 20 via a filter (not shown) installed in the air purifier 40.

The filter is a 13-level or higher HEPA filter that filters out 99.95% or more of 0.3 μm-sized particles, and may completely filter out 0.5 μm-sized droplets contained in contaminated air inside the main body 20.

Accordingly, it can be seen that the performance of the filter is superior to that of a N95 mask (for use by a medical professional) that blocks 95% or more of 0.3 μm-sized particles, which is the highest grade mask according to the Korean Ministry of Food and Drug Safety.

In addition, when the service life of the filter expires, it is replaced with a new filter.

In addition, the blower 45 is a device that communicates with both opposite sides of the main body 20 and serves to selectively introduce external air and discharge air inside the main body 20.

The mobile isolation ward 10 according to the present invention is configured to further include a sensor unit 50 and a controller 70.

First, the sensor unit 50 measures environment-related data inside or outside the main body 20. The data measured by the sensor unit 50 is transmitted to the controller.

In addition, the controller controls an environment inside the main body 20 to provide a customized medical environment optimized for each patient based on the data measured by the sensor unit 50.

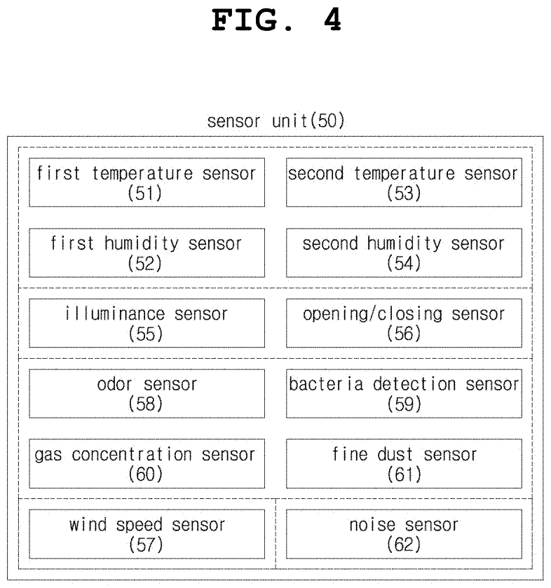

FIG. 4 is a block diagram of the sensor unit 50.

Referring to FIG. 4, the sensor unit 50 is configured to include a first temperature sensor 51, a first humidity sensor 52, a second temperature sensor 53, a second humidity sensor 54, an illuminance sensor 55, an opening/closing sensor 56, a wind speed sensor 57, an odor sensor 58, a bacteria detection sensor 59, a gas concentration sensor 60, a fine dust sensor 61, and a noise sensor 62.

First, the first temperature sensor 51 is provided on one side of the inside of the main body 20 and serves to measure the actual temperature inside the main body 20.

In addition, the first humidity sensor 52 is provided on the other side of the inside of the main body 20 and serves to measure the actual humidity inside the main body 20.

In addition, the second temperature sensor 53 is provided on one side of the outside of the main body 20 and serves to measure the actual temperature outside the main body 20.

In addition, the second humidity sensor 54 is provided on the other side of the outside of the main body 20 and serves to measure the actual humidity outside the main body 20.

In addition, the illuminance sensor 55 measures the actual illuminance inside the main body 20. In this case, the unit of measurement of illuminance is set to “lux.”

In addition, the opening/closing sensor 56 detects whether the door 21 is opened or closed and generates an opening signal indicative of the opening of the door 21 or a closing signal indicative of the closing of the door 21.

In addition, the odor sensor 58 measures the actual concentration of odor-causing material that floats in the inner space of the main body 20 and generates odor.

In addition, the bacteria detection sensor 59 detects the actual concentration of bacteria inside the main body 20.

In addition, the gas concentration sensor 60 measures the actual concentration of carbon dioxide inside the main body 20.

In addition, the fine dust sensor 61 measures the actual concentration of fine dust inside the main body 20.

In addition, the noise sensor 62 measures the actual noise inside the main body 20. In this case, the measurement unit of noise is set to “decibels.”

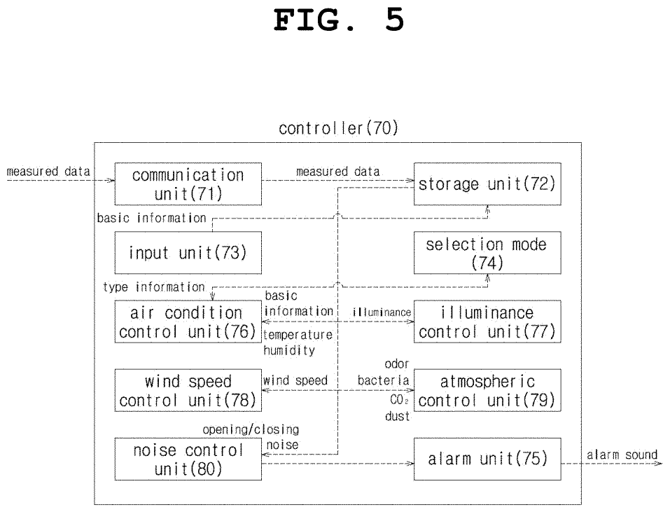

FIG. 5 is a block diagram of the controller 70.

Referring to FIG. 5, the controller 70 is configured to include a communication unit 71, a storage unit 72, an input unit 73, a selection mode 74, an alarm unit 75, an air condition control unit 76, an illuminance control unit 77, a wind speed control unit 78, and an atmospheric control unit 79.

First, the communication unit 71 enables the controller 70, the door 21, the patient monitoring equipment 28, the ceiling air conditioner 30, the air purifier 40, and the blower 45 to communicate with each other. In this case, the communication unit 71 is preferably implemented as a Bluetooth module, which is a short-range communication module.

In addition, a plurality of pieces of data measured by the sensor unit 50 is stored in the storage unit 72. In addition, a plurality of biosignals measured by the patient monitoring equipment 28 is stored in the storage unit 72.

In this case, a set temperature section and a set humidity section refer an appropriate temperature section and an appropriate humidity section inside the main body 20, respectively, that are set to vary according to the temperature or humidity outside the main body 20.

In this case, the basic information of a patient input to the input unit 73, a reference temperature section and reference humidity section for each type of patient, and a set temperature section and set humidity section set according to the external temperature or humidity are stored in the storage unit 72.

In addition, in the selection mode 74, the type of patient suitable for the disease of a patient accommodated in the main body 20 may be selected. For example, the type of patient may be classified as a general patient, a diabetic patient, a high blood pressure patient, an obese patient, a heart disease patient, or an asthma patient.

In addition, the alarm unit 75 generates an alarm sound when an emergency situation occurs.

In addition, the air condition control unit 76 calculates a standard temperature and standard humidity suitable for the patient based on the basic information of the patient input to the input unit 73, the type of patient selected by the selection mode 74, and the actual temperature and actual humidity outside the main body 20, and controls the ceiling air conditioner 30 according to the standard temperature and the standard humidity.

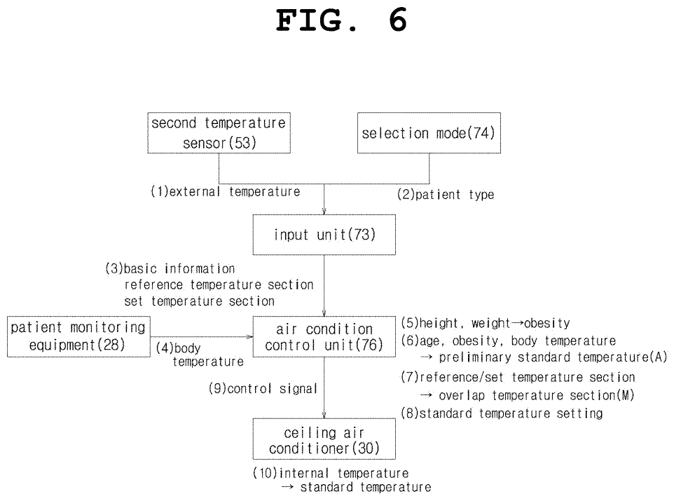

FIG. 6 is a block diagram illustrating a process in which the air condition control unit 76 performs control so that the temperature inside the main body 20 reaches the standard temperature.

Referring to FIG. 6, the air condition control unit 76 calculates the patient's obesity degree according to the patient's height and weight. In addition, the air condition control unit 76 calculates a preliminary standard temperature inside the main body 20 suitable for the patient by taking into consideration the patient's age, obesity, and body temperature in an integrated manner.

More specifically, the air condition control unit 76 calculates the preliminary standard temperature inside the main body 20 as a lower value as the patient's age is younger or the patient's obesity is higher. Furthermore, the air condition control unit 76 calculates the preliminary standard temperature inside the main body 20 as a lower value as the patient's body temperature is higher.

In addition, the air condition control unit 76 calculates an overlap temperature section in which temperature values overlap each other by comparing a reference temperature section based on the type of patient and a set temperature section based on the external temperature.

In addition, when the preliminary standard temperature calculated according to the patient's basic information falls within the overlap temperature section, the air condition control unit 76 sets the preliminary standard temperature as the standard temperature suitable for the patient.

In addition, when the preliminary standard temperature calculated according to the patient's basic information exceeds the overlap temperature section, the air condition control unit 76 sets the upper limit of the overlap temperature section as the standard temperature.

In addition, when the preliminary standard temperature calculated according to the patient's basic information is lower than the overlap temperature section, the air condition control unit 76 sets the lower limit of the overlap temperature section as the standard temperature.

In addition, the air condition control unit 76 controls the ceiling air conditioner 30 to operate until the actual temperature inside the main body 20 reaches the standard temperature.

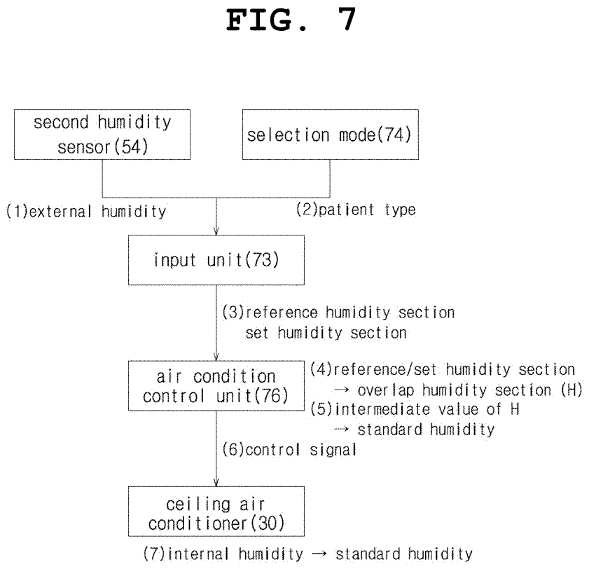

FIG. 7 is a block diagram illustrating a process in which the air condition control unit 76 performs control so that the actual humidity inside the main body 20 reaches the standard humidity.

Referring to FIG. 7, the air condition control unit 76 calculates an overlap humidity section in which humidity values overlap each other by comparing a reference humidity section based on the type of patient and a set humidity section based on the actual external humidity.

In addition, the air condition control unit 76 calculates the average value of the upper and lower limits of the overlap humidity section, sets the average value as a standard humidity, and performs control so that the ceiling air conditioner 30 operates until the actual humidity inside the main body 20 reaches the standard humidity.

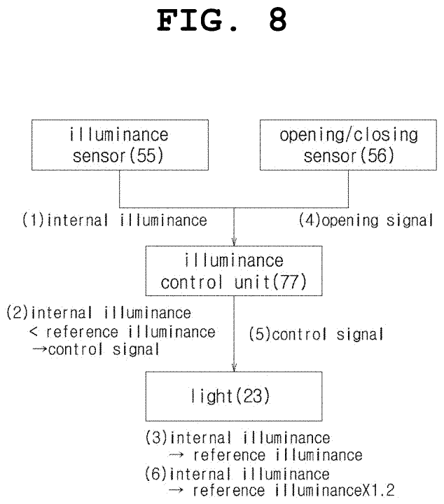

FIG. 8 is a block diagram illustrating a process in which the illuminance control unit 77 performs control so that the actual illuminance inside the main body 30 reaches a reference illuminance.

Referring to FIG. 8, when the actual illuminance inside the main body 20 measured by the illuminance sensor 55 is lower than a preset reference illuminance, the illuminance control unit 77 performs control so that the illuminance of the light 23 increases until the actual illuminance inside the main body 20 reaches the reference illuminance.

In this case, the reference illuminance refers to the illuminance inside the main body 20 that is set to vary according to time. For example, the reference illuminance from 1 p.m. to the time immediately before 2 p.m. may be set to a value higher than that of the reference illuminance from 9 p.m. to the time immediately before 10 p.m.

In addition, in response to an opening signal indicative of the opening of the door 21 generated by the opening/closing sensor 56, the illuminance control unit 77 performs control so that the illuminance of the light 23 increases until the actual illuminance inside the main body 20 becomes 1.2 times the reference illuminance.

For example, when a medical staff member such as a doctor or a nurse arrives at the mobile isolation ward 10, the door 21 is automatically opened. Thereafter, in response to an opening signal indicative of the opening of the door 21, the illuminance controller 77 may perform control so that the illuminance of the light 23 increases until the actual illuminance inside the main body 20 becomes 1.2 times the reference illuminance.

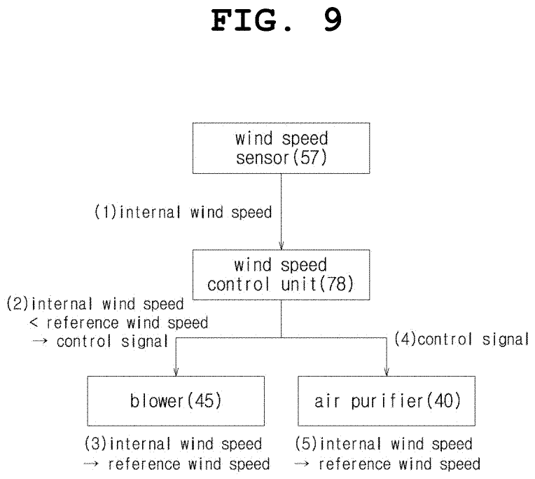

FIG. 9 is a block diagram illustrating a process in which the wind speed control unit 78 performs control so that the actual wind speed inside the main body 20 reaches a reference wind speed.

Referring to FIG. 9, when the actual wind speed inside the main body 20 measured by the wind speed sensor 57 is lower than a preset reference wind speed, the wind speed control unit 78 performs control so that the wind speeds of the air purifier 40 and the blower 45 increase until the actual wind speed inside the main body 20 reaches the reference wind speed.

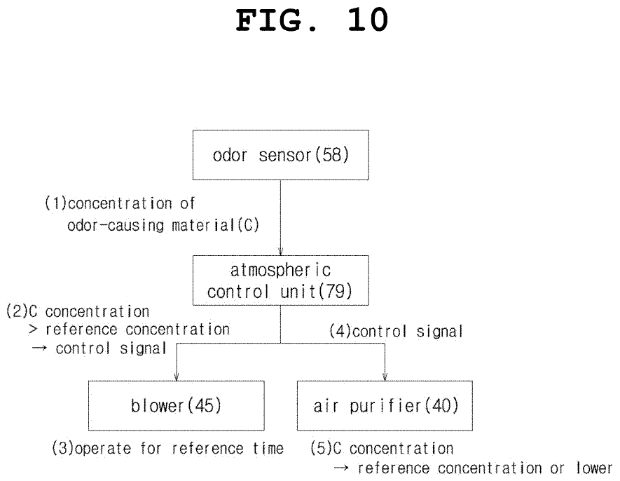

FIG. 10 is a block diagram illustrating a process in which the atmospheric control unit 79 performs control so that the actual concentration of odor-causing material inside the main body 20 becomes equal to or lower than a reference concentration.

Referring to FIG. 10, when the actual concentration of odor-causing material measured by the odor sensor 58 exceeds a preset reference concentration, the atmospheric control unit 79 performs control so that the blower 45 operates for a preset reference time.

In addition, the atmospheric control unit 79 performs control so that the air purifier 40 operates until the actual concentration of the odor-causing material inside the main body 20 decreases to a value equal to or lower than the reference concentration.

In addition, when the actual concentration of bacteria inside the main body 20 detected by the bacteria detection sensor 59 exceeds a preset concentration, the atmospheric control unit 79 performs control so that the air purifier 40 operates until the actual concentration of bacteria in the main body 20 decreases to a value equal to or lower than the set concentration.

Furthermore, when the actual concentration of carbon dioxide inside the main body 20 measured by the gas concentration sensor 60 exceeds a preset upper limit concentration, the atmospheric control unit 79 performs control so that the blower 45 operates until the actual concentration of carbon dioxide inside the main body 20 decreases to a value equal to or lower than the preset upper limit concentration.

Meanwhile, when the actual concentration of fine dust inside the main body 20 measured by the fine dust sensor 61 exceeds a preset limit concentration, the atmospheric control unit 79 performs control so that the air purifier 40 operates until the actual concentration of fine dust inside the main body 20 decreases to a value equal to or lower than the limit concentration.

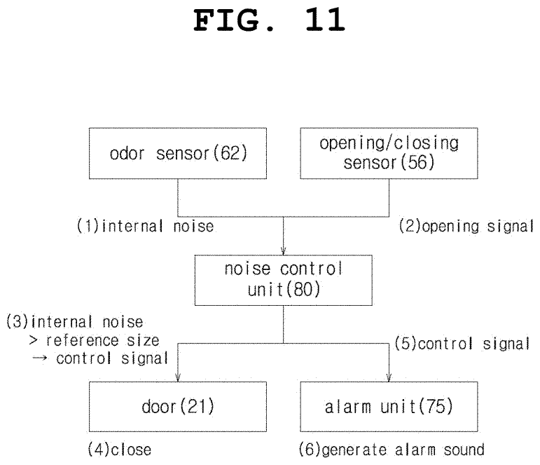

FIG. 11 is a block diagram illustrating a process in which the noise control unit 80 reduces the noise inside the main body 20.

Referring to FIG. 11, when the actual noise inside the main body 20 measured by the noise sensor exceeds a preset reference level and an opening signal indicative of the opening of the door 21 is generated by the opening/closing sensor 56, the noise control unit 80 performs control so that the door 21 is closed and the alarm unit 75 generates an alarm sound.

In the mobile isolation ward 10 according to the present invention, the main body 20 is made of a glass fiber reinforced plastic material in a compact size, so that an effect is achieved in that it has desirable corrosion resistance, heat resistance, and durability and is easy to manufacture, thereby significantly reducing manufacturing time and cost.

Furthermore, in the mobile isolation ward 10 according to the present invention, the inner surface of the main body 20 is surface-treated with an eco-friendly polymer paint, so that an effect is achieved in that diseases such as dermatitis, respiratory diseases, and carcinogenesis may be prevented in advance.

Furthermore, in the mobile isolation ward 10 according to the present invention, the fixed lighting window 22 finished with silicone is provided in the right side of the main body 20, so that an effect is achieved in that the leakage of contaminated air inside the main body 20 to the outside may be suppressed as much as possible.

Furthermore, in the mobile isolation ward 10 according to the present invention, the air purifier 40 equipped with the 13-level or higher HEPA filter 41 that filters out 99.95% or more of 0.3 μm-sized particles is installed on one side of the main body 20, so that an effect is achieved in that 0.5 μm-sized droplets contained in contaminated air inside the main body 20 may be completely filtered out.

Moreover, in the mobile isolation ward 10 according to the present invention, the system for controlling an environment inside the main body 20 is provided according to the personal information, type, external temperature and humidity of a patient, so that an effect is achieved in that a customized medical environment optimized for each patient may be provided.

As described above, the present invention has the main technical spirit of providing the mobile isolation ward 10. The embodiments described above with reference to the drawings are merely examples. The true scope of the present invention is defined based on the attached claims, but will also extend to equivalent embodiments that may be present in various manners.

What is claimed is:

1. A mobile isolation ward comprising:
a main body configured such that a door through which a patient can enter and exit is installed in a front thereof, a fixed lighting window is installed in a right side thereof, and a light is installed at a center of a ceiling thereof;
a ceiling air conditioner installed in a right side of the ceiling of the main body, and configured to control a temperature and a humidity inside the main body;
an air purifier provided on one side of an inside of the main body, and configured to purify contaminated air inside the main body via a filter installed therein; and
a blower communicating with both opposite sides of the main body, and configured to selectively introduce external air and discharge air purified by the air purifier,
wherein junctions of the lighting window and the main body are finished with silicone,
wherein the main body is made of a glass fiber reinforced plastic material, and
wherein the door and the lighting window are made of a transparent acrylic material.

2. The mobile isolation ward of claim 1, wherein a hospital bed configured to allow a patient to lay thereon is provided in a lower side of the inside of the main body,
wherein a storage cabinet is disposed on a left side of a portion over the hospital bed,
wherein a mattress is disposed on a right side of the cabinet, and
wherein the filter is a 13-level or higher high-efficiency particulate air (HEPA) filter.

3. The mobile isolation ward of claim 2, wherein an inner surface of the main body is surface-treated with an eco-friendly polymer paint, and
wherein a plurality of ultraviolet sterilization lamps configured to sterilize the inside of the main body is provided on one side of the inside of the main body.

4. The mobile isolation ward of claim 1, further comprising:
patient monitoring equipment configured to check the patient's condition in real time by continuously monitoring biosignals such as the patient's electrocardiogram, oxygen saturation, body temperature, blood pressure, and respiration rate;
a sensor unit configured to measure environment-related data inside or outside the main body; and
a controller configured to control an internal environment of the main body to provide a customized medical environment optimized for each patient based on data measured by the sensor unit,
wherein the sensor unit comprises:
a first temperature sensor configured to measure an actual temperature inside the main body;
a first humidity sensor configured to measure an actual humidity inside the main body;
a second temperature sensor configured to measure an actual temperature outside the main body;
a second humidity sensor configured to measuring an actual humidity outside the main body;
an illuminance sensor configured to measure an actual illuminance inside the main body;
an opening/closing sensor configured to detect whether the door is opened or closed, and to generate an opening signal or a closing signal;
an odor sensor configured to measure an actual concentration of an odor-causing material floating in an inner space of the main body;
a bacteria detection sensor configured to detect an actual concentration of bacteria inside the main body;
a gas concentration sensor configured to measure an actual concentration of carbon dioxide in the main body;
a fine dust sensor configured to measure an actual concentration of fine dust inside the main body; and
a noise sensor configured to measure an actual noise inside the main body.

5. The mobile isolation ward of claim 4, wherein the controller comprises:
an input unit configured to receive basic information of the patient, a reference temperature section and reference humidity section for each type of patient, a set temperature section based on an actual temperature outside the main body, and a set humidity section based on an actual humidity outside the main body;
a selection mode configured such that the type of patient suitable for a disease of the patient accommodated in the main body is selected;
an alarm unit configured to generate an alarm sound when an emergency situation occurs; and
an air condition control unit configured to calculate a standard temperature and standard humidity suitable for the patient based on the basic information of the patient, the type of patient, and the actual temperature and actual humidity outside the main body, and to control the ceiling air conditioner according to the standard temperature and the standard humidity,
wherein the basic information is a height, weight, and age of the patient, and
wherein the air condition control unit is further configured to:
calculate obesity of the patient based on the height and weight of the patient, and calculate a preliminary standard temperature inside the main body suitable for the patient by taking into consideration the age, obesity, and body temperature of the patient in an integrated manner;
calculate the preliminary standard temperature inside the main body as a lower value as the age of the patient is younger or the obesity of the patient is higher, and calculate the preliminary standard temperature inside the main body as a lower value as the body temperature of the patient is higher;
calculate an overlap temperature section in which temperature values overlap each other by comparing the reference temperature section based on the type of patient and the set temperature section based on the actual temperature outside the main body;
set the preliminary standard temperature as the standard temperature suitable for the patient when the preliminary standard temperature calculated according to the basic information of the patient falls within the overlap temperature section, set an upper limit of the overlap temperature section as the standard temperature when the preliminary standard temperature calculated according to the basic information of the patient exceeds the overlap temperature section, and set a lower limit of the overlap temperature section as the standard temperature when the preliminary standard temperature calculated according to the basic information of the patient is lower than the overlap temperature section; and
perform control so that the ceiling air conditioner operates until the actual temperature inside the main body becomes the standard temperature.

6. The mobile isolation ward of claim 5, wherein the air condition control unit is further configured to:
calculate an overlap humidity section in which humidity values overlap each other by comparing the reference humidity section based on the type of patient and the set humidity section based on the actual humidity outside the main body; and
calculate an average value of upper and lower limits of the overlap humidity section, set the average value as the standard humidity, and perform control so that the ceiling air conditioner operates until the actual humidity inside the main body reaches the standard humidity.

7. The mobile isolation ward of claim 5, wherein the controller comprises:
an illuminance control unit configured to, when the actual illuminance inside the main body measured by the illuminance sensor is lower than a preset reference illuminance, perform control so that an illuminance of the light increases until the actual illuminance inside the main body reaches the reference illuminance;
a wind speed control unit configured to, when the actual wind speed inside the main body measured by the wind speed sensor is lower than a preset reference wind speed, perform control so that wind speeds of the air purifier and the blower increase until the actual wind speed inside the main body reaches the reference wind speed;
an atmospheric control unit configured to, when the actual concentration of odor-causing material measured by the odor sensor exceeds a preset reference concentration, perform control so that the blower operates for a preset reference time and also perform control so that the air purifier operates until the actual concentration of the odor-causing material inside the main body decreases to a value equal to or lower than the reference concentration; and
a noise control unit configured to, when the actual noise inside the main body measured by the noise sensor exceeds a preset reference level and the opening signal indicative of opening of the door is generated by the opening/closing sensor, perform control so that the door is closed and the alarm unit generates an alarm sound, and
wherein the reference illuminance is an illuminance inside the main body that is set to vary according to time.

8. The mobile isolation ward of claim 7, wherein the illuminance control unit is further configured to perform control so that the illuminance of the light increases until the actual illuminance inside the main body becomes 1.2 times the reference illuminance in response to the opening signal indicative of the opening of the door generated by the opening/closing sensor, and
wherein the atmospheric control unit is further configured to:
when the actual concentration of bacteria inside the main body detected by the bacteria detection sensor exceeds a preset concentration, perform control so that the air purifier operates until the actual concentration of bacteria in the main body decreases to a value equal to or lower than the set concentration;
when the actual concentration of carbon dioxide inside the main body measured by the gas concentration sensor exceeds a preset upper limit concentration, perform control so that the blower operates until the actual concentration of carbon dioxide inside the main body decreases to a value equal to or lower than the preset upper limit concentration; and
when the actual concentration of fine dust inside the main body measured by the fine dust sensor exceeds a preset limit concentration, perform control so that the air purifier operates until the actual concentration of fine dust inside the main body decreases to a value equal to or lower than the limit concentration.
